(19) United States
(12) Patent Application Publication
(10) Pub. No.: US 2006/0004366 A1
(43) Pub. Date: Jan. 5, 2006

(54) Pro sleeve

(76) Inventor: Alexis Cerul, San Jose, CA (US)

Correspondence Address: Alexis Cerul, 551 E. Empire St, San Jose, CA, 95112, US

(21) Appl. No.: 11/105,703

(22) Filed: Apr. 15, 2005

Related U.S. Application Data
(63) Continuation of application No. 10/872,796, filed on Jun. 21, 2004, now Pat. No. 6,901,628.

(51) Int. Cl. A61B 17/56

(52) U.S. Cl. 606/73

(57) ABSTRACT

The Pro Sleeve is a tubular steel grommet, with an open channel running its length, which would be placed around a wire or pipe by passing the wire/pipe through the open channel. The channel may be bent closed, and/or locked shut, thereafter the Pro Sleeve should be slid along the wire/pipe and inserted into the hole, drilled through the stud or joist, that the wire/pipe passes through. The Pro Sleeve has anchors on both sides to keep it in place. The anchors, short strips of metal attached to the body, are bent 90 degrees thus securing the Pro Sleeve in the interior of the drilled hole. When the Pro Sleeve is in place it provides a shield around the wire/pipe, by stopping or deflecting any nail or screw coming into the stud or joist, thus protecting the wires and pipes from being compromised.

### CROSS-REFERENCE TO RELATED APPLICATION

This specification discloses some subject matter common to that disclosed and claimed in the co-pending Alexis Cerul application titled “Protector Sleeve” filed Jun. 21, 2004, numbered Ser, No. 10/872,796, (which was a continuation of “Protector Sleeve For Wires And Pipes In Wood Frame Construction” filed Feb. 28, 2002 and thereafter issued as U.S. Pat. No. 6,779,232) and is, therefore, a continuation in part of said applications and patent which I request be herein incorporated by reference.

### STATEMENT REGARDING FEDERALLY SPONSORED RESEARCH OR DEVELOPMENT

Not applicable.

### REFERENCE TO SEQUENCE LISTING, A TABLE OR A COMPUTER PROGRAM LISTING COMPACT DISC APPENDIX

Not applicable.

### BACKGROUND OF THE INVENTION

From a consideration of the cross referenced applications it will be apparent that it primarily pertains to devices that protect wires or pipes from being punctured, ruptured, or otherwise compromised, by nails, screws, or other fasteners, that would be driven into the walls, ceilings, or floors of buildings and other structures constructed by means of wood studs and joists. For example, when a house is built with wood studs there must be holes drilled in those studs so that wires and pipes can reach their final destination. Once the drywall or paneling is attached to the studs one cannot see where the wires or pipes are and one runs the risk of compromising them when hanging or attaching things to the wall, at the point of the studs, with screws or nails that might need to be several inches long to support their load and serve their function.

My prior applications presented, for the reasons disclosed therein, improvements over the prior state of the art “protector plate” and this application discloses variations and improvements over those in at least a couple ways. First, this new Pro Sleeve is capable of being installed after the wire or pipe has already been installed. Whereas my prior specifications required installation of the invention before the wire or pipe is run through the wood, this specification discloses a variation that can be used during remodeling projects when walls, floors or ceilings are opened up and wires or pipes that are already installed once again become accessible. This modified design, as does my second specification but unlike my first, readily lends itself to being produced by taking the shape from a sheet of steel and rolling it into its final form.

Secondly, since the Pro Sleeve disclosed herein has a strip of metal removed from its body and may be squeezed into its final shape by the person installing it, there will be savings in the production process both in materials and machine tooling given the simplified design.

### BRIEF SUMMARY OF THE INVENTION

The Pro Sleeve is a grommet, preferably cylindrical or rectangular, made of steel, that would be located in a hole drilled through a stud or joist in wood frame construction. A preferred embodiment resembles a length of pipe with a ¼ inch strip missing from the length of its body. The missing strip, or gap, forms an opening and allows the Pro Sleeve to be placed around a wire. The Pro Sleeve may then be squeezed so that the ends, where the missing strip was, overlap, butt, or lock together. The Pro Sleeve, substantially or fully encircling the wire, is slid along the wire until it is inserted into the wood hole that the wire passes through. The Pro Sleeve has anchors on both sides to keep it in place. The anchors, short strips of metal attached to the body, which are bent 90 degrees and can have barbs, or holes for a fastener (nail/screw/tack), will allow the body of the Pro Sleeve to rest or be suspended in the hole drilled in the stud. When the Pro Sleeve is in place it shields the interior of the hole that was drilled, thereby protecting the wire or pipe that the hole was drilled for from being punctured by any screw or nail that might be driven through the wall and into the stud. The steel of the Pro Sleeve either stops, or deflects, any nail or screw.

### BRIEF DESCRIPTION OF THE SEVERAL VIEWS OF THE DRAWING

FIG. 1 is a cylindrical Pro Sleeve, presenting its opening at the bottom, with interchangeable anchors, utilizing a hole in the anchors to secure it in place, in final form.

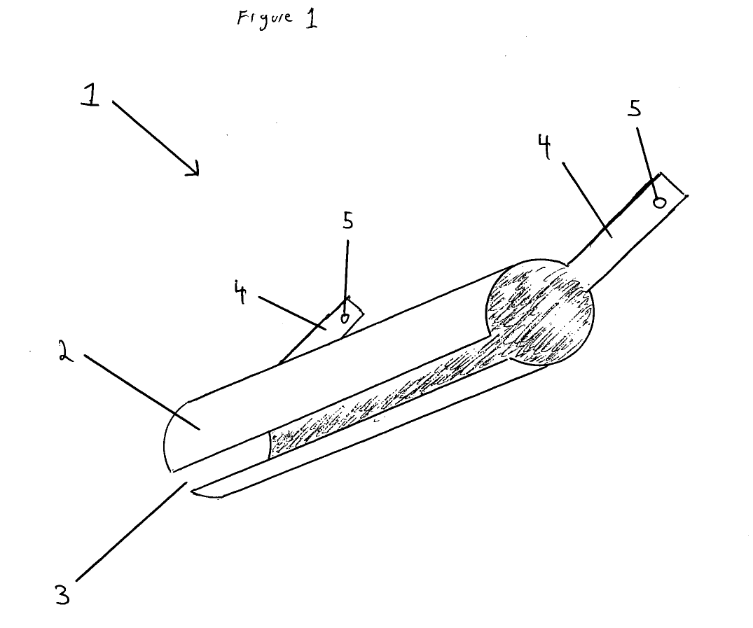

FIG. 2 is a view of the Pro Sleeve looking down through its interior. For this embodiment the open space, which allows installed wires to pass into its interior, is at its bottom.

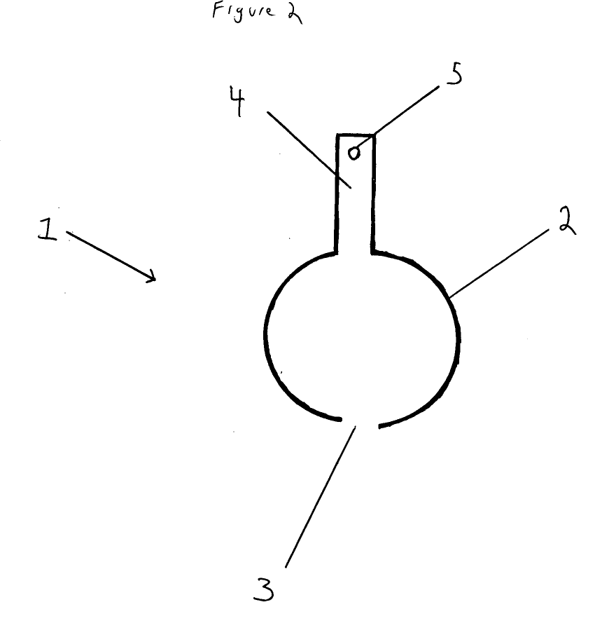

FIG. 3 is the Pro Sleeve of figure two after it has been placed around the wire(s) and squeezed together.

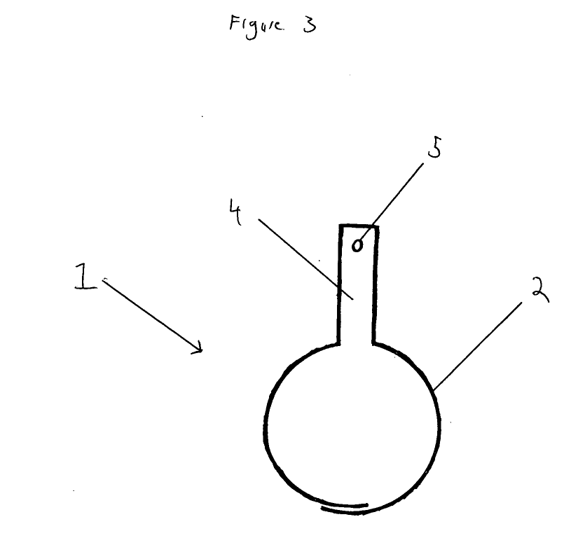

FIG. 4 is an embodiment of the Pro Sleeve with a clasp at its bottom. The forty five degree bends at the opening, projecting in opposite directions, run down the length of the opening in the body.

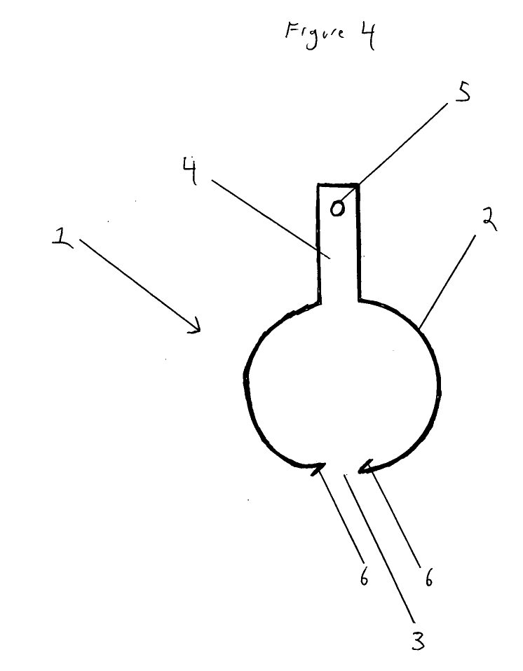

FIG. 5 is the Pro Sleeve of figure four after it has been squeezed together from its sides so that the two hooks of the clasp grip each other and fasten the bottom where the opening used to be.

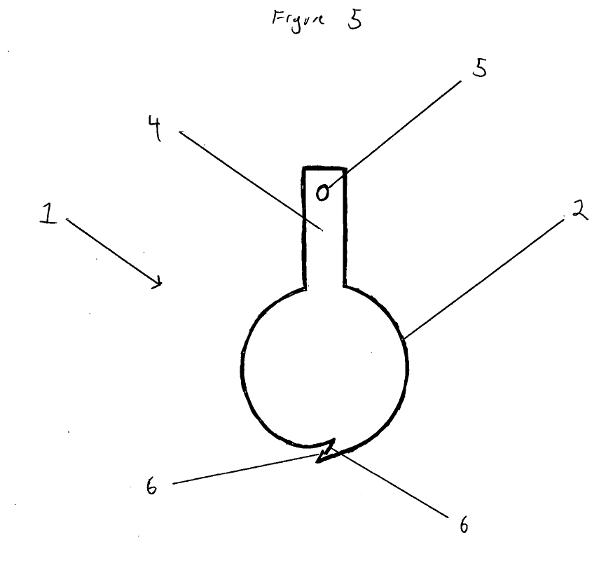

FIG. 6 is a square embodiment of the Pro Sleeve with forty five degree bends to the metal at the opening projecting in the same direction (inward in this case however they could just as easily project outward).

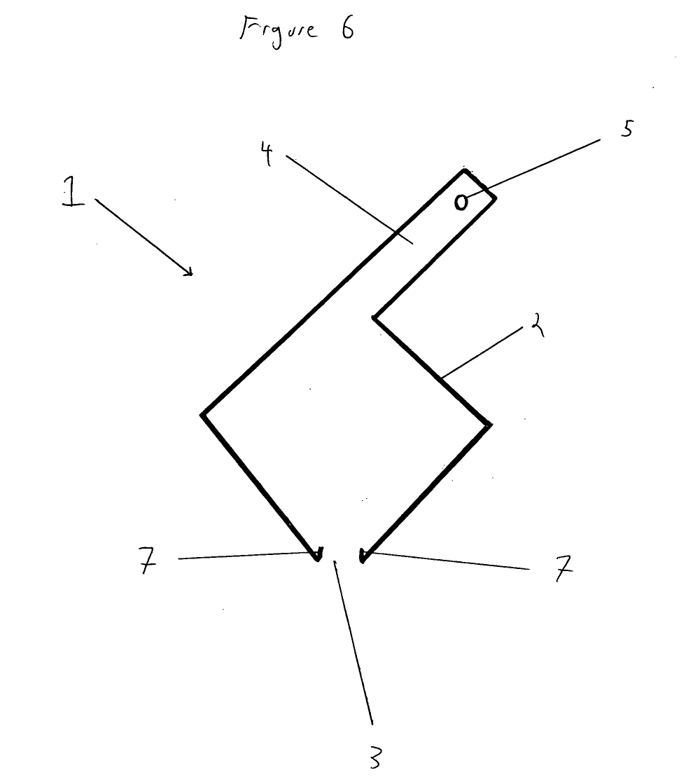

FIG. 7 is the Pro Sleeve of figure six after the sides have been squeezed together. The forty five degree bends at the opening present flat sides to each other at the junction and thereby prevent further deformation.

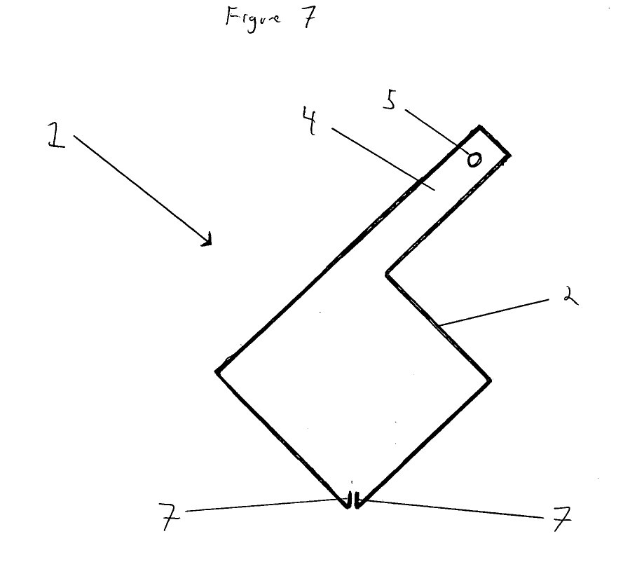

FIG. 8 is an embodiment of the Pro Sleeve with a U shaped channel which would serve as a stop when the edges of the opening butt together, when the Pro Sleeve is squeezed together from its sides, after being placed around the wire(s) or pipe that the Pro Sleeve is to protect.

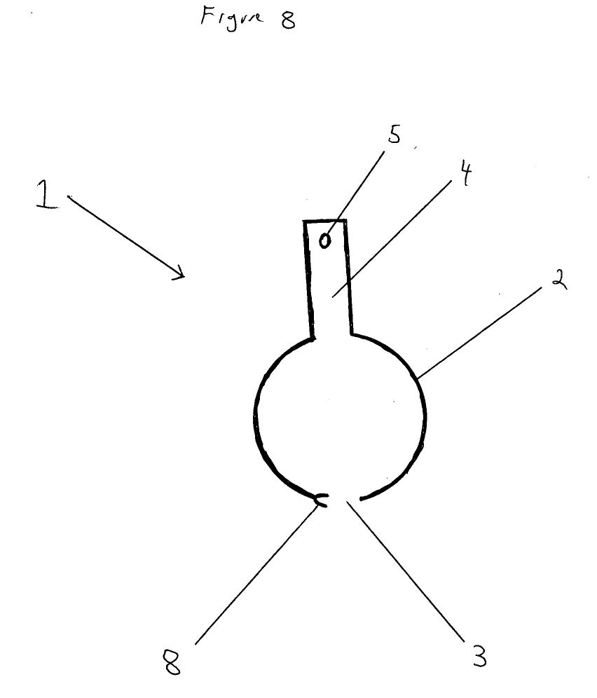

FIG. 9 is an embodiment of the Pro Sleeve with a clasp somewhat like a ball and socket. It clicks into place and locks the opening closed after the Pro Sleeve has been placed around the wire(s) or pipe and squeezed together from its sides.

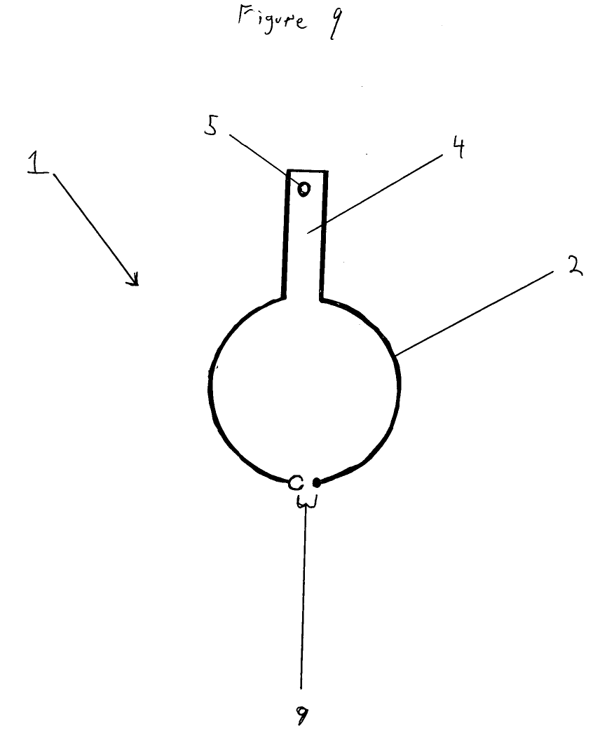

FIG. 10 is a variation of the Pro Sleeve shown at the point in the production process where it has just been cut from a sheet of steel and has not yet been rolled into its cylindrical form. The rectangular protrusions of this embodiment will be given a slightly offset angle so that when squeezed together they will interlock.

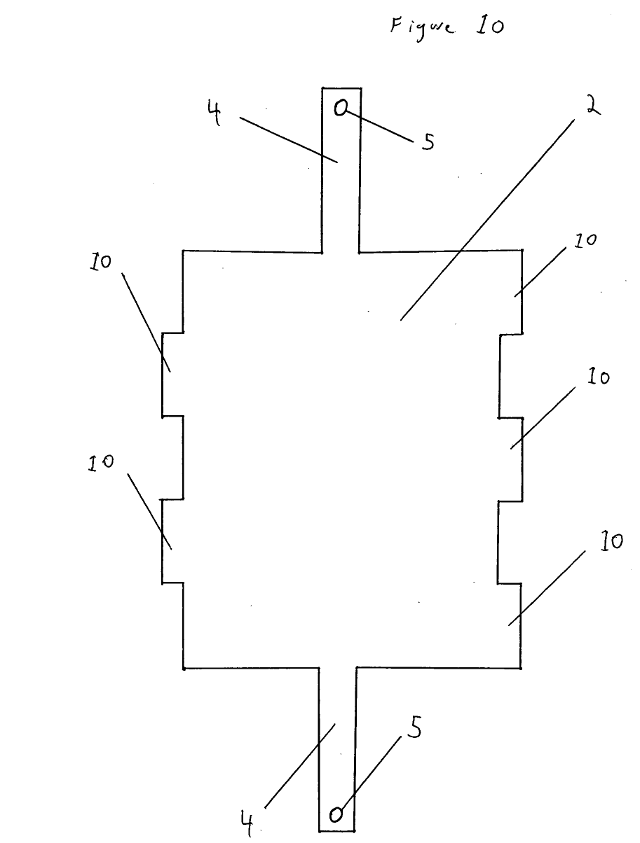

### FIGURE LEGEND

1: An embodiment of the Pro Sleeve.
2: The body of the Pro Sleeve.
3: Opening in the body of the Pro Sleeve which allows it to be placed around wires or pipes that are already installed in wood frame construction.
4: An anchor, initial or final, of the Pro Sleeve.
5: Hole in the anchor for nail mounting.
6: A hook variation of the clasp for closing the opening after installation around the wire(s) or pipe and before mounting in the wood stud or joist.
7: Short stubs or lips made by bending the metal at the Pro Sleeve's opening, forming between a 45 to 135 degree angle, to serve as stops to prevent excessive deformation when installed Pro Sleeve is squeezed together at its opening.
8: U shaped stop to catch other side of Pro Sleeve opening when squeezed shut.
9: Ball and socket variation of the clasp for closing the Pro Sleeve opening after installation around the wire(s) or pipe and before mounting in the wood stud or joist.
10: Rectangular protrusions, or teeth, from the Pro Sleeve's body, at its opening, to be angled and offset so as to interlock when the Pro Sleeve's opening is squeezed closed.

### DETAILED DESCRIPTION OF THE INVENTION

The objective of the invention is to provide a device that can be installed around wires or pipes, after they themselves have been installed, which will act like a shield, or a coat of armor, around them so that there is no risk that they will be damaged by anything that might be driven into the wall, ceiling or floor. In wood frame construction, studs and joists usually present a two inch side and are sixteen inches apart. Wires and pipes run through the walls, ceilings and floors and pass through the studs or joists by means of holes drilled in those studs or joists. Therefore, behind such walls there are large voids of space between the studs. When somebody wants to hang or attach something to the wall they will seek to drive the fastener through the wall material and into the stud. This is because the wood of the stud provides substantial strength and support whereas the relatively thin wall material might not be strong enough and is certainly less desirable for this purpose. If, by stroke of bad luck, the nail or screw goes into the stud at the point where there is a hole with a wire or pipe going through it, the nail or screw, if long enough, can enter the hole and pinch the wire or pipe against the other side of the hole, and then possibly penetrate it, thereby creating a dangerous, or at least undesirable, situation.

The body 2 of the Pro Sleeve 1 is its operative component and it amounts to a hollow tube of steel as long as the depth of the hole drilled in the stud. The Pro Sleeve 1 may, but does not have to, be installed after the wires and pipes have been run through the studs or joists. The Pro Sleeve 1 has a slit, or opening 3, in its body 2 that allows it to be placed around a wire or pipe that is already installed. One simply passes the wire or pipe through the slit/opening 3 so that the wire or pipe is inside the body 2 of the Pro Sleeve 1. The Pro Sleeve 1 can then be slid along the wire and into the hole in the stud or joist. Once the body 2 is in its position inside the stud or joist the Pro Sleeve 1 should be secured into place by bending the anchors 4 into their final position perpendicular to the body 2 and flush with the side of the stud or joist.

After being placed around the wire(s) or pipe(s) it is to protect, the Pro Sleeve 1 is able to be squeezed together from its sides so that the slit/opening 3 closes. A score line, or line of tiny perforations, running the length of the body 2, opposite the slit/opening 3 will facilitate bending it so that the slit/opening 3 closes. The edges of the slit/opening 3 may pass each other and overlap so as to provide full encirclement of the wires or pipe.

A preferred embodiment of the Pro Sleeve 1 has a clasp at its opening 3 which, when the body 2 is squeezed together from its sides so that the slit 3 closes, will lock the opening 3 closed. The clasp can be made by bending an eighth inch lip from the metal all along the edges of the opening to make forty five degree hooks 6. The bends, and thus the hooks 6, should be in opposite directions. When squeezed together the hooks 6 will slide past each other and then, because of the springiness of the body 2 will click and nest into each other forming a tight clasp. Another way to form the clasp is with a ball and socket type mechanism 9. One side of the opening has a cylindrical shaft running down it and the other has a C-shaped groove that the cylindrical shaft fits perfectly into. It should take a little bit of force to get the cylindrical shaft to click into the C-shaped groove so that it does not come apart easily.

Another preferred embodiment has a tail or stub bent from the edge of the body 2 at its opening 3, to form a small, perhaps one eighth inch, lip 7. These lips 7 should point in the same direction so that when the Pro Sleeve 1 is squeezed together the lips 7 meet and the Pro Sleeve 1 cannot be squeezed or deformed any further. The lips 7 act as stops so that the Pro Sleeve 1 achieves a final shape with its opening 3 closed. A variation of this embodiment with a stop has a U-shaped or V-shaped groove 8 on one side that receives and catches the other side of the opening 3 when squeezed together. Yet another variation of this embodiment with a stop has alternating offset teeth 10, possibly rectangular, that fit together so as to prevent further deformation.

Thus assembled around the wire or pipe, and then slid into place in the hole in the stud or joist, the Pro Sleeve 1 serves as a shield, or armor, defending the wire or pipe from everything around it. Particularly, the steel protects the wire or pipe from nails or screws penetrating the wood stud and coming towards its location.

The steel used for the body 2 should be at least, approximately, one sixteenth inches thick as this should be strong enough to withstand penetration, or substantial distortion, by a nail or screw. Since the Pro Sleeve 1 will hang, or rest, loosely in the drilled hole in the stud, it will also serve its purpose by deflecting the nail or screw away from itself. Depending on the size of the drilled hole, as compared to the diameter of the Pro Sleeve's body 2, in any given situation there could be enough room in the hole so that the nail or screw will deviate from its course when it hits the Pro Sleeve 1 rather than stopping. Just as a rock thrown against the ground at an angle will ricochet, whereas a rock thrown against the ground perpendicularly will stop, so too the Pro Sleeve 1 will deflect and divert the path of a nail or screw if the angles allow. In this regard the use of square tubing for the body 2 will be preferred. When made with square tubing, the body 2 of the Pro Sleeve 1 would have to be orientated during installation so as to present forty five degree angles to the parallel sides of the stud.

As noted in passing above, the Pro Sleeve 1 is installed in a wood hole by means of anchors 4 on both sides of its body 2. The anchors 4 are short strips of steel attached to the body 2 and projecting outward from the body's 2 ends. The shape of the anchors 4 would be rectangular and, for a typical two inch by four inch stud application, would be one quarter to one half inches wide and approximately one to two inches long. After the Pro Sleeve 1 has been placed in its hole, and both anchors 4 are bent into place so that they are flush against the stud, the Pro Sleeve 1 will not be able to move out of the hole.

As described above, the anchors 4 are rectangular in shape if examined without regard to the body 2. One of the short sides of the rectangle is attached to the body 2. Near the end of the other short side there may be either a forty five degree notch, a triangularly tapered tip, or a triangular piece at a right angle, which would be bent ninety degrees away from the body 2 so as to form a barb 6. The barb 6 that would be thus formed would be embedded into the wood when the anchor 4 is bent or installed in its appropriate ninety degree fashion.

Alternatively, near the end of the anchor 4 there may be a hole 5, approximately one eighth to one quarter inch in diameter, through which a nail or screw can be used to secure its position.

No matter which anchor 4 variation is used, (hole 5 for fastener, barb, or nothing) the Pro Sleeve 1 will be completely installed when, after insertion in the hole, both anchors 4 are bent ninety degrees and are against the wood. Bending the anchors 4 will only require a few hammer taps. If the anchor 4 is a spiked variety, then maybe an extra hammer tap will be necessary to fully embed the spike into the wood. The Pro Sleeve 1 may come from the factory with one of its anchors 4 already bent ninety degrees. This may be done for the convenience of the installer but it might not be done if it adds substantial production or shipping costs.

Use of a barb, or a fastener through the fastener hole 5, is not mandatory because simply bending the anchors 4 flush against the wood will mean that the Pro Sleeve 1 will not work its way out of the hole or move at all along its axis. The anchors 4 without barbs or fasteners will secure the body 2 in its appropriate place by virtue of friction and the fact that its final shape, modified by the installation process, can not physically move out of the hole. Admittedly, without the barb or fastener the Pro Sleeve 1 is capable of moving more freely within the hole than when a barb or fastener through the fastener hole 5 is used. However, this may be a desirable characteristic. If the Pro Sleeve 1 is capable of some movement within the hole it will mean that if a nail or screw does strike it, the Pro Sleeve 1 will first shift position and, if eventually pinned against the hole wall will present as obtuse an angle as possible and thereby deflect the nail or screw away from it more easily. The Pro Sleeve 1 secured by fasteners or barbs 6 in the anchors 4 will still usually deflect the nail or screw away but there may be situations where the nail or screw will strike at a right angle and then the Pro Sleeve 1 will only protect by virtue of being made of the harder material.

The Pro Sleeve 1 can be made as a unit by performing sheet metal processing using a steel sheet, for example. It can also be made by creating a flat sheet in the shape of, or by cutting or stamping from an existing sheet, the form and then rolling it into its cylindrical shape. Installing the cylindrical Pro Sleeve 1 will usually call for the anchors 4 to be secured vertically and thus the opening 3 would be at the bottom. Given a vertical anchor 4 installation there will be no chance of a nail or screw entering the opening 3 since a nail or screw will not come from such an angle towards the bottom of the hole in the stud or joist.

The exact measurements will vary depending on the application and whatever any local building codes might require. Field or laboratory testing may reveal that something other than one sixteenth of an inch will be necessary or sufficient to withstand penetration or crushing by nails or screws and strong enough to deflect nails or screws away from and around it.

The anchors 4 should be seamlessly connected to the body 2 of the Pro Sleeve 1. Even though the initial anchor 4 and final anchor 4 are described separately they are intended to be part of the same continuous piece of steel.

Although the present invention has been described and illustrated in detail, it is clearly understood that the same is by way of illustration and example only and is not to be taken by way of limitation, the scope of the present invention being limited only by the terms of the appended claims.

1: I claim a grommet/bushing type device comprised of a tubular body having a first end and a second end, an initial anchor at said first end, a final anchor at said second end, and an open channel/slit running between the ends, integrally formed as a unit, made of steel or a similarly hard material, that is installed around a wire or pipe by passing the wire or pipe in a parallel manner through the open channel into the center of the tubular body, the device thereafter being slid along the wire or pipe and into the hole in the stud or joist that the wire or pipe passes through, the purpose of which is to provide a protective shield around the wire or pipe so that it may not be penetrated or compromised by any nail, screw, or sharp object being driven into the stud or joist in the same area, the device being secured in place by its anchors which are comprised of one or more rectangular strips projecting outward from each end of the body, which are thereafter bent away from the body to form a substantially right angle and lay against the stud or joist, so that the anchors are substantially perpendicular to the body, said anchors possibly having a barb, or a hole for a tack, at their far ends, which would allow them to be more securely fastened to the stud or joist.

2: A device as in claim one which, after placement around the wire or pipe and before being slid into place in the stud or joist, has its tubular body squeezed together from its sides so that the open channel closes, the edges of the previously open channel overlapping.

3: I claim a grommet/bushing type device comprised of a tubular body having anchors at its ends, and an open channel/slit running between the ends, integrally formed as a unit, made of steel or a similarly hard material, that is installed around a wire or pipe by passing the wire or pipe in a parallel manner through the open channel into the center of the tubular body, said tubular body thereafter being squeezed together from its sides so that the open channel closes, the edges of the previously open channel locking together, the device thereafter being slid along the wire or pipe and into the hole in the stud or joist that the wire or pipe passes through, the purpose of which is to provide a protective shield around the wire or pipe so that it may not be penetrated or compromised by any nail, screw, or sharp object being driven into the stud or joist in the same area, the device being secured in place by its anchors which are comprised of one or more rectangular strips projecting outward from each end of the body, which are thereafter bent away from the body to form a substantially right angle and lay against the stud or joist, so that the anchors are substantially perpendicular to the body, said anchors possibly having at their far ends a barb, or a hole for a tack, which would allow them to be more securely fastened to the stud or joist.

4: A device as in claim 3 wherein said squeezing is facilitated by a score line, or line of tiny perforations, running the length of the body substantially opposite the channel.

5: A device as in claim 3 wherein said locking is accomplished by a clasp comprising hooks which are formed by bending, approximately forty five degrees, a lip, said lip being approximately one eights of an inch long, along the edge of the open channel, said forty five degree hooks projecting in opposite directions so that when the device is squeezed together the hooks pass each other, and thereafter the tension caused by the squeezing of the body of the device causes the hook ends to spring back and nest into each other.

6: A device as in claim 3 wherein said locking is accomplished by a ball and socket type clasp comprising a solid round lip, like the shaft of a nail, attached to one side of the open channel, and a C-shaped groove, the open end of which runs along the length of the other side of the channel, so that when the device is squeezed it clicks together with the C-shaped groove receiving the cylindrical lip into it and holding the ends together.

7: I claim a grommet/bushing type device comprised of a tubular body having anchors at its ends and an open channel/slit running between the ends, integrally formed as a unit, made of steel or a similarly hard material, that is installed around a wire or pipe by passing the wire or pipe in a parallel manner through the open channel into the center of the tubular body, said tubular body thereafter being squeezed together from its sides so that the open channel closes, the edges of the previously open channel butting together, the device thereafter being slid along the wire or pipe and into the hole in the stud or joist that the wire or pipe passes through, the purpose of said device being to provide a protective shield around the wire or pipe so that it may not be penetrated or compromised by any nail, screw, or sharp object being driven into the stud or joist in the same area, the device being secured in place by its anchors which are comprised of one or more rectangular strips projecting outward from each end of the body, which are thereafter bent away from the body to form a substantially right angle and lay against the stud or joist, so that the anchors are substantially perpendicular to the body, said anchors possibly having at their ends furthest from the body a barb, or a hole for a tack, which would allow them to be more securely fastened to the stud or joist.

8: A device as in claim 7 wherein said squeezing is facilitated by a score line, or line of tiny perforations, running the length of the body substantially opposite the channel.

9: A device as in claim 7 wherein said ends butting together is facilitated by a U-shaped, or V-shaped, groove on one side of the open channel, said U-shaped, or V-shaped, groove presenting its open top in the direction of the other side of the channel so that when the device is squeezed the U-shaped, or V-shaped, groove receives the other side of the open channel into it.

10: A device as in claim 7 wherein said ends butting together is facilitated by lips, approximately one eights of an inch long, along the edge of the open channel, which are formed by bending the edges of the open channel, in the case of a cylindrical embodiment ninety degrees, in the same direction, so that the lips present their sides parallel to each other and thus serve to stop the device from deforming further when squeezed together.

11: A rectangular embodiment of the device specified in claim 7, said channel occurring at the corner/intersection of the two sides opposite the anchors, wherein said ends butting together is facilitated by lips, approximately one eights of an inch long, along the edge of the open channel, which are formed by bending the edges of the open channel either forty five degrees or one hundred and thirty five degrees, in the same direction, so that the lips present their sides parallel to each other and thus serve to stop the device from deforming further when squeezed together.

12: A device as in claim 7 wherein said ends butting together is facilitated by alternating offset teeth, possibly rectangular, along the edge of the open channel, that fit together when the device is squeezed together from the sides of its body.
